(19) United States
(12) Patent Application Publication
(10) Pub. No.: US 2024/0207496 A1
(43) Pub. Date: Jun. 27, 2024

(54) DUAL CHAMBER MEDICAL FLUID CONTAINER WITH VENTED SUPPLY LINE CAP AND METHOD THEREFOR

(71) Applicants: Baxter International Inc., Deerfield, IL (US); Baxter Healthcare SA, Glattpark (Opfikon) (CH)

(72) Inventors: Sumana Roychowdhury, Bangalore (IN); Pushpa Yadav, District-Ghazipur UP (IN); Vandana Sakhare, Wagholi (IN)

(21) Appl. No.: 18/393,164

(22) Filed: Dec. 21, 2023

(30) Foreign Application Priority Data
Dec. 26, 2022 (IN) 202241075524

(51) Int. Cl. A61M 1/16; A61M 39/10; A61M 39/20

(52) U.S. Cl. CPC A61M 1/1668; A61M 39/10; A61M 39/20; A61M 2039/1083; A61M 2039/1088; A61M 2205/7536

(57) ABSTRACT

A medical fluid container tubing assembly includes a medical fluid container, a first luer connector, a tube extending from the medical fluid container and terminating at the first luer connector, and a cap fitted onto the first luer connector. The cap includes a hydrophobic filter positioned and arranged to allow air to aseptically enter the tube to equalize pressure inside and outside of the tube. The medical fluid container tubing assembly also includes a supply line extending from a medical fluid destination and terminating with a second luer connector. The second luer connector is configured to mate with the first luer connector when the cap is removed from the first luer connector.

### PRIORITY CLAIM

This application claims priority to and the benefit as a non-provisional application of Indian Provisional Patent Application No. 202241075524, filed Dec. 26, 2022, the entire contents of which are hereby incorporated by reference and relied upon.

### TECHNICAL FIELD

The present disclosure relates generally to medical fluid treatments, and in particular to medical fluid treatments using premade or bagged medical fluid.

### BACKGROUND

Due to various causes, a person's renal system can fail. Renal failure produces several physiological derangements. It is no longer possible to balance water and minerals or to excrete daily metabolic load. Toxic end products of metabolism, such as, urea, creatinine, uric acid, and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins, and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

Any of the above treatment modalities may operate with premade, e.g., bagged, solutions. Bagged solutions are typical for any type of PD (CAPD or APD). A bagged solution may also be used for HD, especially HHD (see for example U.S. Pat. No. 8,029,454 assigned to the assignee of the present application). Continuous renal replacement therapy (“CRRT”) is an acute form of HD, HF, or HDF and typically uses bagged dialysis fluids. Dual chamber bagged solutions are also provided, where different solution components are separated until the time of use.

Premade, e.g., bagged, solutions for any of the above modalities are typically sterilized after filling and then capped to maintain the medical fluid in a sterilized condition until use. There are different ways to access the sterilized solutions at the time of use. One way is to spike the connector at the time of use, establishing medical fluid flow between the bag and a use point, such as a patient or disposable cassette. Another way, which is very common with PD is to use a breakable frangible. The patient or caregiver bends and snaps open the breakable frangible to thereafter allow fluid flow. A further way is to make a luer connection, which is a known connection that involves threading mating luer connectors together to form a liquid-tight seal between the connectors.

Regardless of the type of connection made, a bag tube is typically provided, which extends from the bag to the connector, such that there is play or room for the operator to grasp and manipulate the connector while making the fluid-tight connection to a mating connector. It has been found that steam sterilization of the bagged solution causes the bag tube to collapse. Here, because the steam outside the bag tube is at a higher pressure than the air inside the bag tube, air is pushed from the tube into the bag, causing the tube to collapse. If the tube does not reopen after steam sterilization, then the bag tube is occluded at the time the solution bag is connected for use.

There is accordingly a need for an improved apparatus and associated methodology for the steam sterilization of bagged dialysis treatment solutions. There is also a need for dual chamber bagged dialysis treatment solutions to be opened in a safe way, such that a properly mixed solution reaches the patient.

### SUMMARY

The present disclosure involves the use of a vented medical fluid supply line cap for use with a solution container or bag that is operable with any type of dialysis treatment including any type of peritoneal dialysis (“PD”) treatment, hemodialysis (“HD”) treatment, hemofiltration (“HF”) treatment, hemodiafiltration (“HDF”) treatment, or continuous renal replacement therapy (“CRRT”) treatment. It should be appreciated that the vented medical fluid supply line cap may be used in any type of medical treatment having a bagged or otherwise stored medical fluid, which needs to be opened aseptically for use, and which is steam sterilized. The vented medical fluid supply line cap may therefore be used additionally with any type of bagged medical infusion or intravenous fluid, saline, lactated ringers, etc.

The vented medical fluid supply line cap is in one embodiment configured to cap a luer connector. It should be appreciated, however, that the medical fluid supply line cap does not have to cap a luer connector and may instead cap a different type of connector. In any case, the cap caps a short line or tube, which may also be called a pigtail, where the short line or tube extends from a medical fluid supply container, e.g., a flexible bag. The flexible bag may be a single chamber bag holding a fully mixed medical fluid or be a multi-chamber bag having one or more peel seals separating medical fluid components that need to be isolated until the time of use. bbbb

The vented medical fluid supply line cap is in one embodiment formed, e.g., molded as one piece, from a polymer, such as, polyetherimide (“PEI”), polyethersulfone (“PES”), polyamide/nylon (“PA”), acrylonitrile butadiene styrene (“ABS”), polycarbonate (“PC”) polyvinylchloride (“PVC”), nylon, polyether ether ketone (“PEEK”) and/or a thermoplastic elastomer, such as one marketed under the tradename Hytrel®. The medical fluid container or bag and the bag tube (which may be a short tube or pigtail) may be made of PVC or other suitable medically safe material.

When the vented medical fluid supply line cap is configured to cap a luer connector, the cap is one embodiment configured to have female luer features, while the luer connector is a male luer connector. In alternative embodiments, the cap may be provided with male luer features, while the luer connector is a female luer connector. When the vented medical fluid supply line cap is configured as having female luer features, the cap includes a body having an inner port and an outer shroud. An inner surface of the inner port, in one embodiment, includes a female luer taper that forms an interference fit with an outer surface of an inner male luer port of the mating male luer connector. An outer surface of the inner port is in one embodiment provided with a plurality of ribs, e.g., extending longitudinally along the outer wall. The ribs form a secondary interference fit with female threads provided on an inner surface of an outer shroud of the mating male luer connector. The female threads threadingly connect to male threads of a female luer connector to establish medical fluid flow when the vented medical fluid supply line cap is removed from the male luer connector.

An inner surface of the outer shroud is sized in one embodiment to provide a slight amount of clearance with an outer surface of the outer shroud of the mating male luer connector. The clearance allows the interference fit between the inner surface of the inner port of the cap and the outer surface of the inner male luer port of the mating male luer connector to be the primary interference fit that prevents medical fluid from leaking out of the vented medical fluid supply line cap of the present disclosure.

In one embodiment, the vented medical fluid supply line cap is translated onto and off of the mating male luer connector with the interference fit holding the cap onto the male luer connector. In an alternative embodiment, the plurality of ribs provided on the outer surface of the inner port of the cap are replaced with male luer threads that threadingly connect with the female luer threads provided on the inner surface of the outer shroud of the mating male luer connector. Here, the cap threads onto and off of the mating male luer connector in the same manner as the female luer connector used to establish medical fluid flow. In either situation (translated or threaded), an outer surface of the outer shroud of the cap is formed with a plurality of ribs, e.g., longitudinally extending ribs, which aid the user in translating or threading the cap onto or off of the mating male luer connector.

In an embodiment, the lumen formed by the inner surface of the inner port extends through the body to and through a circular distal end portion of the body. An enlarged diameter cavity is provided at the distal end of the body in one. The cavity provides a location to attach a vent, such as a hydrophobic membrane filter, within the cavity, such that the opening through the body of the cap is covered. The hydrophobic filter or vent allows air but not medical fluid to flow through the filter or vent, into or out of the body of the cap. Also, air flowing into the body of the cap is aseptically filtered via the hydrophobic filter or vent. In an embodiment, the hydrophobic filter or vent includes a 0.2 micron polytetrafluoroethylene (“PTFE”) hydrophobic membrane having a polyester substrate. The hydrophobic filter or vent may be sealed around its outer diameter to the cavity of the body via heat sealing, ultrasonic sealing or solvent bonding.

When a medical fluid container or bag filled with medical fluid and having a bag tube (e.g., short tube or pigtail) extending from the container, which is capped by the vented cap of the present disclosure is steam sterilized, the hydrophobic filter or vent prevents the short tube or pigtail from collapsing. Steam sterilization typically involves many medical fluid containers or bags being placed within an autoclave and steam sterilized at the same time. The external environment around the medical fluid container or bag is filled with steam, raising the pressure in the autoclave to above ambient pressure (e.g., 15-30 psi (1.0-2.0 bar)) as the temperature within the autoclave may reach 121° C. (250° F.). Without the vented cap of the present disclosure, the short tube or pigtail in many instances collapses under the raised external pressure, wherein the air inside the short tube or pigtail is pushed into the medical fluid container or bag. Heating the thermoplastic tube or pigtail to the sterilization temperature causes it to become somewhat tacky. The collapsed tube thereby tends to stick closed to itself even after removal from the autoclave and cooling. At the time of use, the collapsed tube may become an impediment to good medical fluid, e.g., PD fluid, flow from the medical fluid container or bag to a desired treatment destination, e.g., a PD machine or cycler or to the patient's peritoneal cavity for continuous ambulatory peritoneal dialysis (“CAPD”).

It is accordingly expressly contemplated to provide an improved method for steam sterilizing medical fluid containers, such as PD fluid bags, in which the vented medical fluid supply line cap allows pressurized air from the surrounding autoclave atmosphere to enter the cap and the small tube or pigtail. The pressurized air entering the small tube or pigtail equalizes the pressure on either side of the tube, preventing the tube from collapsing. Also, the air heated to the sterilization temperature, e.g., 121° C. (250° F.), entering the small tube and a portion of the container or bag through the hydrophobic filter or vent brings the sterilization temperature directly to the surfaces contacted (including a portion of the medical fluid held within the container), such that the heat does not have to conduct through the tube and container or bag walls. In this manner, sterilization and sterilization efficiency are improved.

In light of the disclosure set forth herein, and without limiting the disclosure in any way, in a first aspect, which may be combined with any other aspect described herein, or portion thereof, a medical fluid container tubing assembly includes a medical fluid container, a first luer connector, a tube extending from the medical fluid container and terminating at the first luer connector, and a cap fitted onto the first luer connector. The cap includes a hydrophobic filter positioned and arranged to allow air to aseptically enter the tube to equalize pressure inside and outside of the tube. The medical fluid container tubing assembly also includes a supply line extending from a medical fluid destination and terminating with a second luer connector. The second luer connector is configured to mate with the first luer connector when the cap is removed from the first luer connector.

In a second aspect, which may be combined with any other aspect described herein, or portion thereof, the medical fluid container is a multi-chamber medical fluid container including at least one peel seal separating at least two chambers.

In a third aspect, which may be combined with any other aspect described herein, or portion thereof, the first luer connector is a male luer connector and the second luer connector is a female luer connector.

In a fourth aspect, which may be combined with any other aspect described herein, or portion thereof, the second luer connector is configured to connect to a connector of a medical fluid machine to form part of a disinfection loop.

In a fifth aspect, which may be combined with any other aspect described herein, or portion thereof, the supply line is configured at its medical fluid destination end to be connected fluidically within a medical fluid machine or to a disposable cassette operated by the medical fluid machine.

In a sixth aspect, which may be combined with any other aspect described herein, or portion thereof, the cap and the first luer connector are configured such that the cap translates onto and off of the first luer connector.

In a seventh aspect, which may be combined with any other aspect described herein, or portion thereof, the cap and the first luer connector are configured such that the cap threads onto and off of the first luer connector.

In an eighth aspect, which may be combined with any other aspect described herein, or portion thereof, the first luer connector includes a luer port, and the cap includes a port sized to provide an interference fit with the luer port of the first luer connector.

In a ninth aspect, which may be combined with any other aspect described herein, or portion thereof, the luer port is a male luer port, and an inner surface of the port of the cap is sized and shaped to provide an interference fit with an outer surface of the male luer port.

In a tenth aspect, which may be combined with any other aspect described herein, or portion thereof, the first luer connector includes an outer shroud, and an outer surface of the port of the cap includes at least one rib sized to provide a second interference fit with an inner surface of the outer shroud of the first luer connector.

In an eleventh aspect, which may be combined with any other aspect described herein, or portion thereof, the first luer connector includes a first outer shroud, the cap includes a second outer shroud, and the first and second outer shrouds are sized such that a clearance space exists between an outer surface of the first outer shroud and an inner surface of the second outer shroud.

In a twelfth aspect, which may be combined with any other aspect described herein, or portion thereof, an outer surface of the second outer shroud includes at least one rib for grasping the cap to connect and remove the cap from the first luer connector.

In a thirteenth aspect, which may be combined with any other aspect described herein, or portion thereof, the cap defines a lumen that communicates fluidly with the tube, and the hydrophobic filter covers and opening formed by the lumen.

In a fourteenth aspect, which may be combined with any other aspect described herein, or portion thereof, the cap and the first luer connector are configured such that a maximum force needed to remove the cap from the first luer connector is 37 Newtons.

In a fifteenth aspect, which may be combined with any other aspect described herein, or portion thereof, a tubing assembly includes a first luer connector and a cap fitted onto the first luer connector. The cap includes a hydrophobic filter positioned and arranged to allow air to aseptically enter a tube to equalize pressure inside and outside of the tube. The tubing assembly also includes a supply line extending from a medical fluid destination and terminating with a second luer connector. The second luer connector is configured to mate with the first luer connector when the cap is removed from the first luer connector.

In a sixteenth aspect, which may be combined with any other aspect described herein, or portion thereof, the first luer connector includes a luer port, and the cap includes a port sized to provide an interference fit with the luer port of the first luer connector.

In a seventeenth aspect, which may be combined with any other aspect described herein, or portion thereof, the luer port is a male luer port, and an inner surface of the port of the cap is sized and shaped to provide an interference fit with an outer surface of the male luer port.

In an eighteenth aspect, which may be combined with any other aspect described herein, or portion thereof, the first luer connector includes an outer shroud, and an outer surface of the port of the cap includes at least one rib sized to provide a second interference fit with an inner surface of the outer shroud of the first luer connector.

In a nineteenth aspect, which may be combined with any other aspect described herein, or portion thereof, the first luer connector includes a first outer shroud, the cap includes a second outer shroud, and the first and second outer shrouds are sized such that a clearance space exists between an outer surface of the first outer shroud and an inner surface of the second outer shroud.

In a twentieth aspect, which may be combined with any other aspect described herein, or portion thereof, an outer surface of the second outer shroud includes at least one rib for grasping the cap to connect and remove the cap from the first luer connector.

In a twenty-first aspect, any of the features, functionality and alternatives described in connection with any one or more of FIGS. 1 to 11 may be combined with any of the features, functionality and alternatives described in connection with any other of FIGS. 1 to 11.

In light of the above aspects and the present disclosure set forth herein, it is accordingly an advantage of the present disclosure to provide a medical fluid supply line cap that is vented.

It is another advantage of the present disclosure to provide a medical fluid supply line cap that prevents the line from collapsing during steam sterilization.

It is a further advantage of the present disclosure to provide a medical fluid supply line cap that may be used with many different medical fluids.

It is yet another advantage of the present disclosure to provide an improved method for steam sterilizing medical fluid containers, such as PD fluid supply bags.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a fluid flow schematic of one embodiment for a medical fluid, e.g., PD fluid, system that is set for treatment.

FIG. 2 is a fluid flow schematic of one embodiment for a medical fluid, e.g., PD fluid, system that is set for disinfection.

FIG. 3 is a sectioned elevation section view of one embodiment of a medical fluid container tubing assembly of the present disclosure including a container tube, mating male luer connector and a vented medical fluid supply line cap.

FIG. 4 is a perspective view of one embodiment of the medical fluid container tubing assembly of the present disclosure including the container tube, mating male luer connector and vented medical fluid supply line cap.

FIG. 5 is a perspective view of one embodiment of the vented medical fluid supply line cap of the present disclosure.

FIG. 6 is a sectioned perspective view of one embodiment of the vented medical fluid supply line cap of the present disclosure.

FIG. 7 is a sectioned elevation view of one embodiment of the vented medical fluid supply line cap just prior to being applied to the mating male luer connector.

FIG. 8 is a sectioned elevation view of one embodiment of the vented medical fluid supply line cap applied to the mating male luer connector.

FIG. 9 is a sectioned elevation view of detail A of FIG. 8.

FIG. 10 is a sectioned elevation view of detail B of FIG. 8.

FIG. 11 is a perspective view of one embodiment of the medical fluid container tubing assembly of the present disclosure, including the medical fluid container, container tube connected to the male luer connector, vented medical fluid supply line cap removed, and the mating female luer connected to a medical fluid supply line.

### DETAILED DESCRIPTION

### System Overview

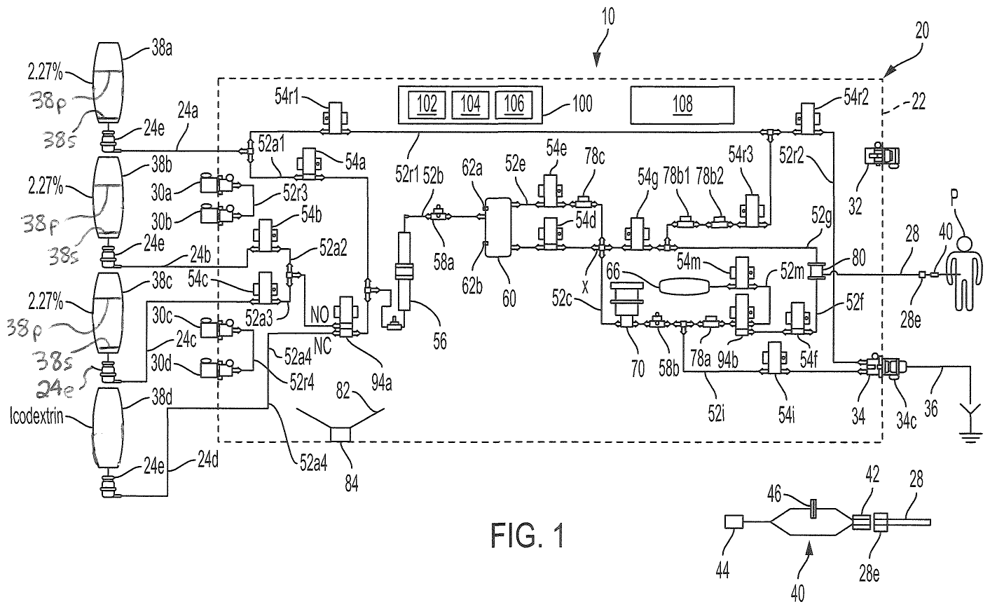

Referring now to the drawings and in particular to FIG. 1, a medical system operable with the multi-chamber medical fluid container tubing assembly 110 described below is illustrated via peritoneal dialysis (“PD”) system 10. System 10 includes a medical machine, such as a PD machine or cycler 20 and a control unit 100 having one or more processor 102, one or more memory 104, video controller 106 and user interface 108. User interface 108 may alternatively or additionally be a remote user interface, e.g., via a tablet or smartphone. Control unit 100 may also include a transceiver and a wired or wireless connection to a network (not illustrated), e.g., the internet, for sending treatment data to and receiving prescription instructions/changes from a doctor's or clinician's server interfacing with a doctor's or clinician's computer. Control unit 100 in an embodiment controls all electrical fluid flow and heating components of system 10 and receives outputs from all sensors of system 10. System 10 in the illustrated embodiment includes durable and reusable components that contact fresh and used PD fluid, which necessitates that PD machine or cycler 20 be disinfected between treatments, e.g., via heat disinfection.

System 10 in FIG. 1 includes an inline resistive heater 56, reusable supply lines or tubes 52al to 52a4 and 52b, air trap 60 operating with respective upper and lower level sensors 62a and 62b, air trap valve 54d, vent valve 54e located along vent line 52e, reusable line or tubing 52c, PD fluid pump 70, temperature sensors 58a and 58b, pressure sensors 78a, 78b1, 78b2 and 78c, reusable patient tubing or lines 52f and 52g having respective valves 54f and 54g, reusable dual lumen patient line 28, a spring-actuated hose reel 80 for retracting patient line 28, reusable drain tubing or line 52i extending to drain line connector 34 and having a drain line valve 54i, and reusable recirculation tubing or lines 52r1 and 52r2 operating with respective disinfection valves 54r1 and 54r2. A third recirculation or disinfection tubing or line 52r3 extends between disinfection or PD fluid line connectors 30a and 30b for use during disinfection. A fourth recirculation or disinfection tubing or line 52r4 extends between disinfection connectors 30c and 30d for use during disinfection.

System 10 further includes medical fluid, e.g., PD fluid, containers or bags 38a to 38c (e.g., holding the same or different formulations of PD fluid), which connect to distal end connector 24e of reusable PD fluid lines 24a to 24c, respectively. System 10d further includes a fourth PD fluid container or bag 38d that connects to a distal end connector 24e of reusable PD fluid line 24d. Fourth PD fluid container or bag 38d may hold the same or different type (e.g., icodextrin) of PD fluid than provided in PD fluid containers or bags 38a to 38c. Reusable PD fluid lines 24a to 24d extend in one embodiment through apertures (not illustrated) defined or provided by housing 22 of cycler 20.

System 10 in the illustrated embodiment includes four disinfection or PD fluid line connectors 30a to 30d for connecting to distal end connectors 24e of reusable PD fluid lines 24a to 24d, respectively, during disinfection. System 10 also provides a patient line connector 32 that includes an internal lumen, e.g., a U-shaped lumen, which for disinfection directs fresh or used dialysis fluid from one PD fluid lumen of a connected distal end 28e of reusable dual lumen patient line 28 into the other PD fluid lumen. Reusable supply tubing or lines 52al to 52a4 communicate with reusable supply lines 24a to 24d, respectively. Reusable supply tubing or lines 52al to 52a3 operate with valves 54a to 54c, respectively, to allow PD fluid from a desired PD fluid container or bag 38a to 38c to be pulled into cycler 20. Three-way valve 94a in the illustrated example allows for control unit 100 to select between (i) 2.27% (or other) glucose dialysis fluid from container or bag 38b or 38c and (ii) icodextrin from container or bag 38d. In the illustrated embodiment, icodextrin from container or bag 38d is connected to the normally closed port of three-way valve 94a.

System 10 is constructed in one embodiment such that drain line 52i during a patient fill is fluidly connected downstream from PD fluid pump 70. In this manner, if drain valve 54i fails or somehow leaks during the patient fill of patient P, fresh PD fluid is pushed down disposable drain line 36 instead of used PD fluid potentially being pulled into pump 70. Disposable drain line 36 is in one embodiment removed for disinfection, wherein drain line connector 34 is capped via a cap 34c to form a closed disinfection loop. PD fluid pump 70 may be an inherently accurate pump, such as a piston pump, or a less accurate pump, such as a gear pump that operates in cooperation with a flowmeter (not illustrated) to control fresh and used PD fluid flowrate and volume.

System 10 may further include a leak detection pan 82 located at the bottom of housing 22 of cycler 20 and a corresponding leak detection sensor 84 outputting to control unit 100. In the illustrated example, system 10 is provided with an additional pressure sensor 78c located upstream of PD fluid pump 70, which allows for the measurement of the suction pressure of pump 70 to help control unit 100 more accurately determine pump volume. Additional pressure sensor 78c in the illustrated embodiment is located along vent line 52e, which may be filled with air or a mixture of air and PD fluid, but which should nevertheless be at the same negative pressure as PD fluid located within PD fluid line 52c.

System 10 in the example of FIG. 1 includes redundant pressure sensors 78b1 and 78b2, the output of one of which is used for pump control, as discussed herein, while the output of the other pressure sensor is a safety or watchdog output to make sure the control pressure sensor is reading accurately. Pressure sensors 78b1 and 78b2 are located along a line including a third recirculation valve 54r3. System 10 may further employ one or more cross, marked via an X in FIG. 1, which may (i) reduce the overall amount and volume of the internal, reusable tubing. (ii) reduce the number of valves needed, and (iii) allow the portion of the fluid circuitry shared by both fresh and used PD fluid to be minimized.

System 10 in the example of FIG. 1 further includes a source of acid, such as a citric acid container or bag 66. Citric acid container or bag 66 is in selective fluid communication with second three-way valve 94b via a citric acid valve 54m located along a citric acid line 52m. Citric acid line 52m is connected in one embodiment to the normally closed port of second three-way valve 94b, so as to provide redundant valves between disinfectant container or bag 66 and the PD fluid circuit during treatment. The redundant valves ensure that no citric acid reaches the treatment fluid lines during treatment. Citric acid is used instead during disinfection.

Control unit 100 in an embodiment uses feedback from any one or more of pressure sensors 78a to 78c to enable PD machine 20 to deliver fresh, heated PD fluid to the patient at, for example, 14 kPa (2.0 psig) or higher. The pressure feedback is used to enable PD machine 20 to remove used PD fluid or effluent from the patient at, for example, between −5 kPa (−0.73 psig) and −15 kPa (−2.2 psig), such as −9 kPa (−1.3 psig) or higher (more negative). The pressure feedback may be used in a proportional, integral, derivative (“PID”) pressure routine for pumping fresh and used PD fluid at a desired positive or negative pressure.

Inline resistive heater 56 under control of control unit 100 is capable of heating fresh PD fluid to body temperature, e.g., 37° C., for delivery to patient P at a desired flowrate. Control unit 100 in an embodiment uses feedback from temperature sensor 58a in a PID temperature routine for pumping fresh PD fluid to patient P at a desired temperature. The control and operation of inline resistive heater 56 for heat disinfection is discussed in detail below:

FIG. 1 also illustrates that system 10 includes and uses a disposable filter set 40, which communicates fluidly with the fresh and used PD fluid lumens of dual lumen patient line 28. Disposable filter set 40 includes a disposable connector 42 that connects to a distal end 28e of reusable patient line 28. Disposable filter set 40 also includes a connector 44 that connects to the patient's transfer set. Disposable filter set 40 further includes a sterilizing grade filter membrane 46 that further filters fresh PD fluid. Disposable filter set 40 is provided in one embodiment as a last chance filter for PD machine 20, which has been heat disinfected between treatments. Any pathogens that may remain after disinfection, albeit unlikely, are filtered from the PD fluid via the sterilizing grade filter membrane 46 of disposable filter set 40.

FIG. 1 illustrates system 10 setup for treatment with PD fluid containers or bags 38a to 38d connected via reusable, flexible PD fluid lines 24a to 24d, respectively. Dual lumen patient line 28 is connected to patient P via disposable filter set 40. Disposable drain line 36 is connected to drain line connector 34. In FIG. 1, PD machine or cycler 20 of system 10 is configured to perform multiple patient drains, patient fills, patient dwells, and a priming procedure, as part of or in preparation for treatment.

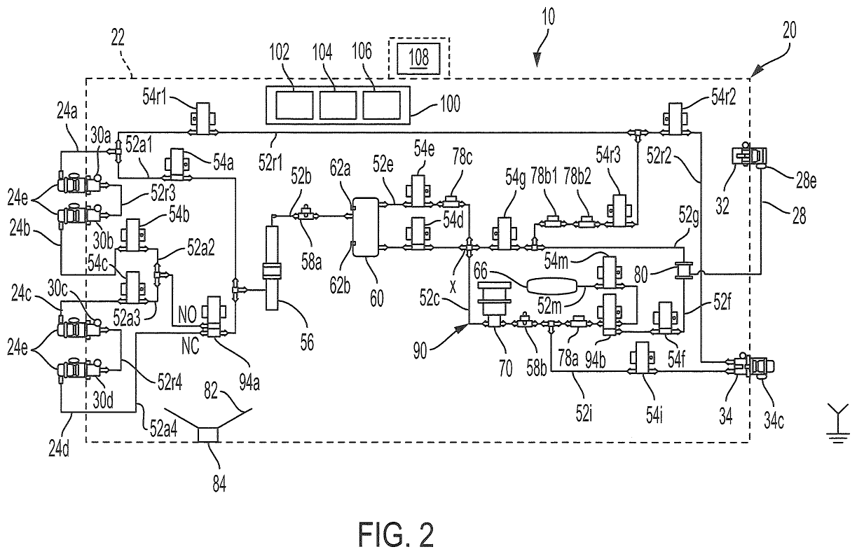

FIG. 2 illustrates system 10 in a disinfection mode. PD fluid containers or bags 38a to 38d are removed and flexible PD fluid lines 24a to 24d are plugged instead in a sealed manner into disinfection or PD fluid line connectors 30a to 30d, respectively. Reusable dual lumen patient line 28 is disconnected from disposable filter set 40 (which is discarded), and distal end 28e of dual lumen patient line 28 is plugged sealingly into patient line connector 32. Disposable drain line 36 is removed from drain line connector 34 and discarded. Drain line connector 34 is capped via cap 34c to form a closed disinfection loop 90. PD machine or cycler 20 of system 10 in FIG. 2 is configured to perform a disinfection sequence, e.g., a heat disinfection sequence in which fresh PD fluid is heated via inline heater 56 to a disinfection temperature of, e.g., 75° C. or greater. PD fluid pump 70 circulates the heated PD fluid closed disinfection loop 90 for an amount of time needed to properly disinfect the fluid components and lines of the disinfection loop.

### Multi-Chamber Medical Fluid Container Assembly

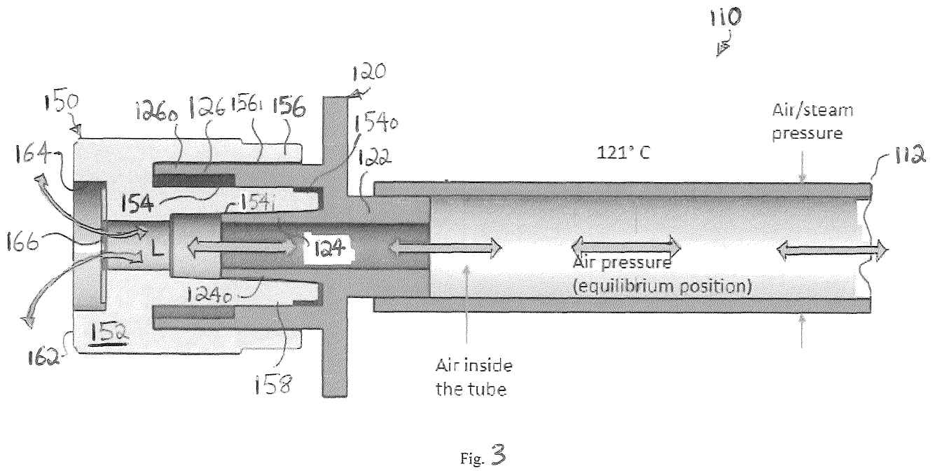

Referring now to FIGS. 3 and 4, an embodiment of a medical fluid container assembly 110 of the present disclosure is illustrated, and which includes a container tube 112, a mating male luer connector 120 and a vented medical fluid supply line cap 150. Container tube 112 extends from one of medical fluid containers or bags 38a to 38d, one or more of which may be a multi-chamber medical fluid container or bag (see FIGS. 1 and 11), wherein containers or bags 38a to 38d are considered as part of medical fluid container tubing assembly 110. Medical fluid containers or bags 38a to 38d are operable with any type of dialysis treatment using bagged dialysis fluid, including any type of peritoneal dialysis (“PD”) treatment, hemodialysis (“HD”) treatment, hemofiltration (“HF”) treatment, hemodiafiltration (“HDF”) treatment or continuous renal replacement therapy (“CRRT”) treatment. It should be appreciated that vented medical fluid supply line cap 150 may be used in any type of medical treatment having a bagged or otherwise stored medical fluid, which needs to be opened aseptically for use, and which is steam sterilized. Vented medical fluid supply line cap 150 may therefore be used additionally with any type of bagged medical infusion or intravenous fluid, saline, lactated ringers, etc.

Vented medical fluid supply line cap 150 is in one embodiment configured to cap a luer connector, such as male luer connector 120. It should be appreciated however that medical fluid supply line cap 150 does not have to cap a luer connector and may instead cap a different type of connector, such as a connector that is spiked to allow medical fluid flow or a connector having a spike that spikes a mating connector to allow medical fluid flow. In any case, cap 150) in the illustrated embodiment caps a short line or tube 112, which may also be called a pigtail, wherein the short line or tube extends from the medical fluid supply container 38a to 38d, e.g., a flexible bag, for a distance of less than one meter. Flexible bag 38a to 38d may be a single chamber bag holding a fully mixed medical fluid or be a multi-chamber bag (FIGS. 1 ad 11) having one or more peel seal(s) 38p. 38s separating medical fluid components that need to be isolated until the time of use. Short line or tube 112 may be heat sealed, ultrasonically sealed or solvent bonded to male luer connector 120.

Vented medical fluid supply line cap 150 is in one embodiment formed, e.g., molded as one piece, from a polymer, such as, polyetherimide (“PEI”), polyethersulfone (“PES”), polyamide/nylon (“PA”), acrylonitrile butadiene styrene (“ABS”), polycarbonate (“PC”) polyvinylchloride (“PVC”), nylon, polyether ether ketone (“PEEK”) and/or a thermoplastic elastomer, such as one marketed under the tradename Hytrel®. Medical fluid container or bag 38a to 38d and short tube or pigtail 112 may be made of PVC or other suitable medically safe material.

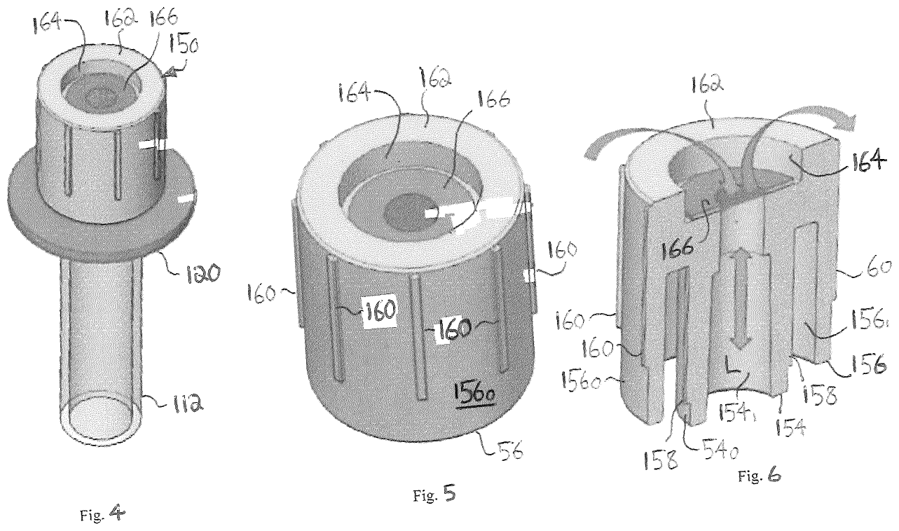

FIGS. 3, 7, 8 and 9 illustrate that where vented medical fluid supply line cap 150 is configured to cap a luer connector, the cap is one embodiment configured to have female luer features, while mating luer connector 120 is a male luer connector. Here, mating luer connector 120 is configured to connect to the distal end distal end connector 24e of one of reusable PD fluid lines 24a to 24d shown in FIGS. 1 and 2. Distal end connectors 24e of reusable PD fluid lines 24a to 24d are here accordingly female luer connectors. In alternative embodiments, cap 150) may be provided with male luer features, while mating luer connector 120 is a female luer connector and distal end connectors 24e of reusable PD fluid lines 24a to 24d are instead male luer connectors.

Where the vented medical fluid supply line cap is configured as having female luer features as illustrated in FIGS. 3, 7, 8 and 9, cap 150 includes a body 152 having or defining an inner port 154 and an outer shroud 156. Mating luer connector 120 in turn includes a body 122 having or defining an inner male luer port 124 and an outer shroud 126. FIGS. 8 and 9 highlight that an inner surface 154i of inner port 154 in one embodiment includes or defines a female luer taper (e.g., a five to ten degree taper—change as needed), which forms an interference fit with an outer surface 1240 of inner male luer port 124 of mating male luer connector 120. FIGS. 6, 7 and 8 highlight that an outer surface 1540 of inner port 154 is in one embodiment provided with a plurality of ribs 158, e.g., extending longitudinally along outer wall 1540. Ribs 158 form a secondary interference fit with female threads provided on an inner surface 126i of outer shroud 126 of mating male luer connector 120. The female threads threadingly connect to male threads of a female luer distal end connector 24e (FIGS. 1 and 11) to establish medical fluid flow after vented medical fluid supply line cap 150 has been removed from male luer connector 120.

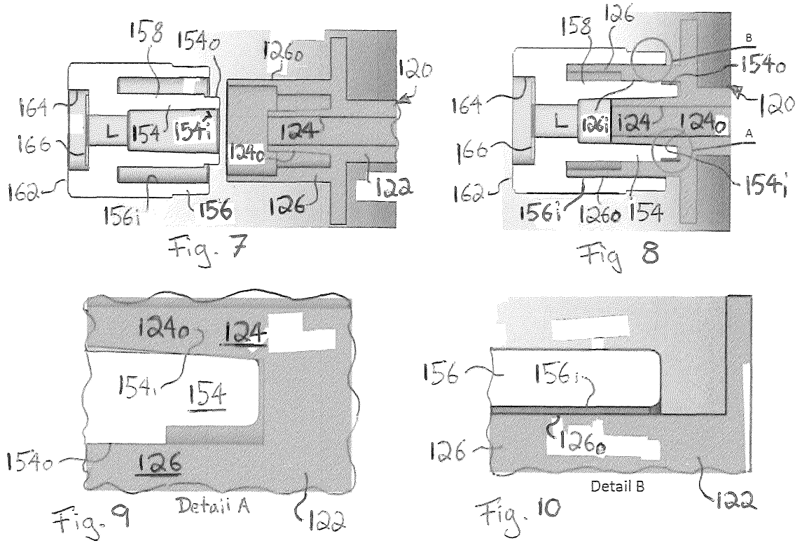

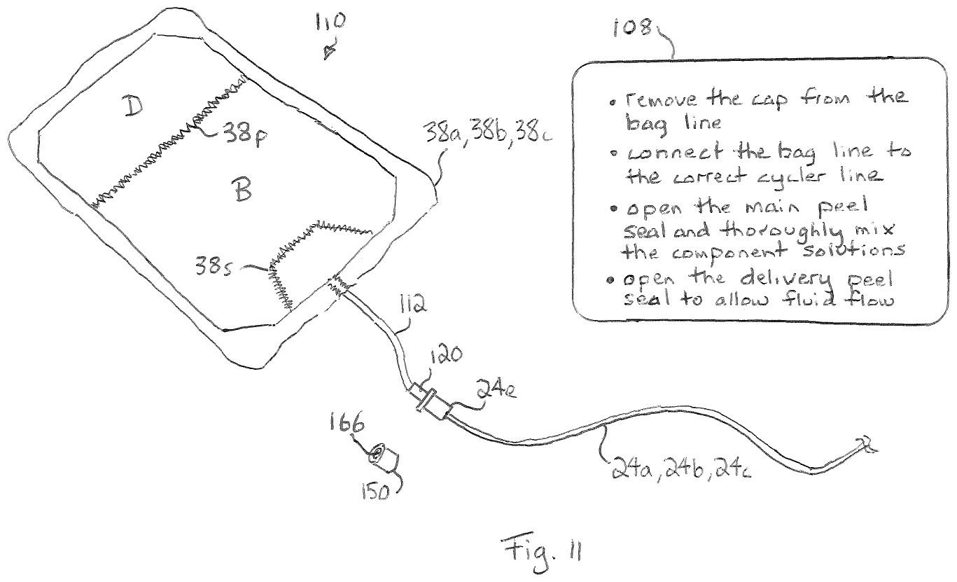

FIGS. 3, 9 and 10 highlight that an inner surface 156i of outer shroud 156 of vented medical fluid supply line cap 150 is sized in one embodiment to provide a slight amount of clearance space with an outer surface 1260 of outer shroud 126 of mating male luer connector 120. The clearance space allows the interference fit between inner surface 154i of inner port 154 of cap 150 and outer surface 1240 of inner male luer port 124 of mating male luer connector 120 to be the primary interference fit that prevents medical fluid from leaking out of vented medical fluid supply line cap 150 when placed or fitted onto mating male luer connector 120.

In one embodiment, vented medical fluid supply line cap 150 is translated onto and off of mating male luer connector 120 with the interference fits described above holding the cap onto the male luer connector. Table 1 below shows that in one embodiment a maximum translational removal force is set to 37 Newtons (“N”). Five different tests each confirmed that the configuration of vented medical fluid supply line cap 150 illustrated herein met the force removal goal:

TABLE 1

| | | | Pull |
|---|---|---|---|
| | | Sample | Force (N) |
| Pull force test of the cap | vented medical fluid | sample#1 | 27.3 |
| from Luer (pull force | supply line cap 50 | sample#2 | 31.4 |
| measured by tensile | shall have a maximum | sample#3 | 33.0 |
| testing equipment with | removal force from | sample#4 | 26.7 |
| load speed = 400 | mating male luer | sample#5 | 35.8 |
| mm/min) | connector 20 of 37 N. | | |

In an alternative embodiment, the plurality of ribs 158 provided on outer surface 1540 of inner port 154 of cap 150 are replaced with male luer threads that threadingly connect with the female luer threads provided on inner surface 126i of outer shroud 126 of mating male luer connector 120. Here, cap 150 threads onto (making the primary interference fit) and off of the mating male luer connector 120 in the same manner as the female luer distal end connector 24e uses to establish medical fluid flow. In either situation (translated or threaded), FIGS. 5 and 6 illustrate that an outer surface 1560 of outer shroud 156 of cap 150 is formed with a plurality of ribs 160, e.g., longitudinally extending ribs, which aid the user in translating or threading cap 150 onto or off of the mating male luer connector 120.

FIGS. 3, 6, 7 and 8 illustrate that in an embodiment, a lumen L formed by inner surface 154i of inner port 154 of vented medical fluid supply line cap 150 extends through body 152, to and through a circular distal end portion 162 of the body. FIGS. 3 to 8 illustrate that an enlarged diameter cavity 164 is provided at the distal end of body 152 in one embodiment. Cavity 164 provides a location to attach a vent 166, such as a hydrophobic membrane filter, within cavity 164, such that the opening or lumen L through body 152 of cap 150 is covered. In the illustrated embodiment, cavity 164 is cylindrically shaped while hydrophobic filter or vent 166 is circular. Hydrophobic filter or vent 166 allows air but not medical fluid to flow through the filter or vent, into or out of body 152 of cap 150. Also, air flowing into body 152 of cap 150 is aseptically filtered via hydrophobic filter or vent 166. In an embodiment, hydrophobic filter or vent 166 includes a 0.2 micron polytetrafluoroethylene (“PTFE”) hydrophobic membrane having a polyester substrate. Hydrophobic filter or vent 166 may be sealed around its outer diameter to cavity 164 of body 152 via heat sealing, ultrasonic sealing or solvent bonding.

When medical fluid container or bag 38a to 38d filled with medical fluid and having a short tube or pigtail 112 extending from the container, which is capped by vented cap 150 of the present disclosure, is steam sterilized, hydrophobic filter or vent 166 prevents short tube or pigtail 112 from collapsing. Steam sterilization typically involves many medical fluid containers or bags being placed within an autoclave and steam sterilized at the same time. The external environment around the medical fluid container or bag is filled with steam, raising the temperature and pressure inside the autoclave to above ambient pressure (e.g., 15-30 psi (1.0-2.0 bar)) as the temperature within the autoclave may reach 121° C. (250° F.). Without vented cap 150 of the present disclosure, short tube or pigtail 112 in many instances collapses under the raised external pressure, wherein the air inside short tube or pigtail 112 is pushed into the medical fluid container or bag. Heating the thermoplastic tube or pigtail 112 to the sterilization temperature causes it to become somewhat tacky. The collapsed tube thereby tends to stick closed to itself even after removal from the autoclave and cooling. At the time of use, the collapsed tube may become an impediment to good medical fluid, e.g., PD fluid, flow from medical fluid container or bag 38a to 38d to a desired treatment destination, e.g., PD machine or cycler 20 or to the patient's peritoneal cavity for continuous ambulatory peritoneal dialysis (“CAPD”).

The airflow arrows of FIGS. 3 and 6 illustrate that it is expressly contemplated to provide an improved method for the steam sterilizing medical fluid containers 38a to 38d, such as PD fluid bags, in which vented medical fluid supply line cap 150 allows pressurized air from the surrounding autoclave atmosphere to enter the cap and small tube or pigtail 112. Pressurized air entering small tube or pigtail 112 equalizes the pressure on either side of the tube, preventing the tube from collapsing. Also, the air heated to the sterilization temperature, e.g., 121° C. (250° F.), entering small tube 112 and a portion of the container or bag through hydrophobic filter or vent 166 brings the sterilization temperature directly to the inner surfaces of medical fluid container tubing assembly 110, including tube 112 and possibly even to a portion of the medical fluid held within the container, such that the heat does not have to conduct through the tube 112 and container or bag walls. In this manner, heat sterilization and heat sterilization efficiency are improved.

FIG. 11 illustrates medical fluid container tubing assembly 110 with vented medical fluid supply line cap 150 removed and mating male luer connector 120 connected to female luer distal end connector 24e of one of reusable PD fluid lines 24a to 24d. Short tube or pigtail 112 extends from male luer connector 120 to medical fluid, such as PD fluid, container or bag 38a to 38c. In the illustrated embodiment, containers or bags 38a to 38c are dual chamber bags that include a first or primary peel seal 38p that separates a buffer solution chamber (marked with a “B”) from a dextrose solution chamber (marked with a “D”). The buffer solution chamber B is larger than the dextrose solution chamber D. The buffer solution chamber is bigger and holds a buffer solution having a pH of around 9.0, while the dextrose solution has a pH of around 3.5. The two solutions are stored separately from each other to prevent particulates from forming when mixed. After the first or primary peel seal 38p is opened by the patient or caregiver, and the component solutions are evenly mixed, the resulting solution has a physiologically neutral pH of around 7.2 and is ready to be delivered to the patient.

Dual chamber bags 38a to 38c are also provided with a second or secondary peel seal 38s, which prevents buffer solution alone from flowing through short tube or pigtail 112, male luer connector 120, female luer distal end connector 24e of one of reusable PD fluid lines 24a to 24d, to PD machine 20 or the patient. After the buffer and dextrose component solutions are properly mixed, the resulting solution may be delivered to the patient. Thoroughly

FIG. 11 also illustrates user interface 108 for PD machine 20, which is shown providing an audio, visual or audiovisual message to the patient or caregiver for properly connecting and opening dual chamber bags 38a to 38c (icodextrin bag 38d is not a dual chamber bag). In FIG. 11, the patient or caregiver is first instructed to remove medical fluid supply line cap 150 from mating male luer connector 120. Second, the patient or caregiver is instructed to connect male luer connector 120 to the female luer distal end connector 24e of one of reusable PD fluid lines 24a to 24c, which is shown as having been completed in FIG. 11. As illustrated in FIGS. 1 and 2, PD fluid lines 24a to 24c extend so as to communicate fluidly with the inner tubing of PD machine 20. Third, the patient or caregiver is instructed to open first or main peel seal 38p and thoroughly mix the buffer and dextrose component solutions. Fourth, the patient or caregiver is instructed to open second or secondary (“delivery”) peel seal 38s to allow fully mixed PD fluid to be removed from dual chamber bags 38a to 38c and used for treatment.

It should be understood that various changes and modifications to the presently preferred embodiments described herein will be apparent to those skilled in the art. It is therefore intended that any or all of such changes and modifications may be covered by the appended claims. For example, while fluid lines 24a to 24d are described as being reusable PD fluid lines extending within PD machine 20 to connect to internal tubing, PD fluid lines 24a to 24d may extend instead to a disposable PD fluid (or other medical fluid) cassette operated by the PD machine (or other type of medical fluid treatment machine). Fluid lines 24a to 24d may accordingly be disposable and be used for any type of medical treatment described herein.

The invention is claimed as follows:

1. A medical fluid container tubing assembly comprising:
a medical fluid container;
a first luer connector;
a tube extending from the medical fluid container and terminating at the first luer connector;
a cap fitted onto the first luer connector, the cap including a hydrophobic filter positioned and arranged to allow air to aseptically enter the tube to equalize pressure inside and outside of the tube; and
a supply line extending from a medical fluid destination and terminating with a second luer connector, the second luer connector configured to mate with the first luer connector when the cap is removed from the first luer connector.

2. The medical fluid container tubing assembly of claim 1, wherein the medical fluid container is a multi-chamber medical fluid container including at least one peel seal separating at least two chambers.

3. The medical fluid container tubing assembly of claim 1, wherein the first luer connector is a male luer connector and the second luer connector is a female luer connector.

4. The medical fluid container tubing assembly of claim 1, wherein the second luer connector is configured to connect to a connector of a medical fluid machine to form part of a disinfection loop.

5. The medical fluid container tubing assembly of claim 1, wherein the supply line is configured at its medical fluid destination end to be connected fluidically within a medical fluid machine or to a disposable cassette operated by the medical fluid machine.

6. The medical fluid container tubing assembly of claim 1, wherein the cap and the first luer connector are configured such that the cap translates onto and off of the first luer connector.

7. The medical fluid container tubing assembly of claim 1, wherein the cap and the first luer connector are configured such that the cap threads onto and off of the first luer connector.

8. The medical fluid container tubing assembly of claim 1, wherein the first luer connector includes a luer port, and wherein the cap includes a port sized to provide an interference fit with the luer port of the first luer connector.

9. The medical fluid container tubing assembly of claim 8, wherein the luer port is a male luer port, and wherein an inner surface of the port of the cap is sized and shaped to provide an interference fit with an outer surface of the male luer port.

10. The medical fluid container tubing assembly of claim 8, wherein the first luer connector includes an outer shroud, and wherein an outer surface of the port of the cap includes at least one rib sized to provide a second interference fit with an inner surface of the outer shroud of the first luer connector.

11. The medical fluid container tubing assembly of claim 1, wherein the first luer connector includes a first outer shroud, the cap includes a second outer shroud, and wherein the first and second outer shrouds are sized such that a clearance space exists between an outer surface of the first outer shroud and an inner surface of the second outer shroud.

12. The medical fluid container tubing assembly of claim 11, wherein an outer surface of the second outer shroud includes at least one rib for grasping the cap to connect and remove the cap from the first luer connector.

13. The medical fluid container tubing assembly of claim 1, wherein the cap defines a lumen that communicates fluidly with the tube, and wherein the hydrophobic filter covers and opening formed by the lumen.

14. The medical fluid container tubing assembly of claim 1, wherein the cap and the first luer connector are configured such that a maximum force needed to remove the cap from the first luer connector is 37 Newtons.

15. A tubing assembly comprising:
a first luer connector;
a cap fitted onto the first luer connector, the cap including a hydrophobic filter positioned and arranged to allow air to aseptically enter a tube to equalize pressure inside and outside of the tube; and
a supply line extending from a medical fluid destination and terminating with a second luer connector, the second luer connector configured to mate with the first luer connector when the cap is removed from the first luer connector.

16. The tubing assembly of claim 15, wherein the first luer connector includes a luer port, and wherein the cap includes a port sized to provide an interference fit with the luer port of the first luer connector.

17. The tubing assembly of claim 16, wherein the luer port is a male luer port, and wherein an inner surface of the port of the cap is sized and shaped to provide an interference fit with an outer surface of the male luer port.

18. The tubing assembly of claim 16, wherein the first luer connector includes an outer shroud, and wherein an outer surface of the port of the cap includes at least one rib sized to provide a second interference fit with an inner surface of the outer shroud of the first luer connector.

19. The tubing assembly of claim 15, wherein the first luer connector includes a first outer shroud, the cap includes a second outer shroud, and wherein the first and second outer shrouds are sized such that a clearance space exists between an outer surface of the first outer shroud and an inner surface of the second outer shroud.

20. The tubing assembly of claim 19, wherein an outer surface of the second outer shroud includes at least one rib for grasping the cap to connect and remove the cap from the first luer connector.
